(19) United States
(12) Patent Application Publication
(10) Pub. No.: US 2016/0324612 A1
(43) Pub. Date: Nov. 10, 2016

(54) MATERIAL MANAGEMENT APPARATUS

(71) Applicant: ROSE STUDIOS, INC., Bellevue, WA (US)

(72) Inventor: Chung-Cheng HSIEH, New Taipei City (TW)

(21) Appl. No.: 14/705,858

(22) Filed: May 6, 2015

(51) Int. Cl. A61C 19/02; A61C 8/00; G06K 7/12; G06Q 10/08; G06K 7/10; G06K 19/077

(52) U.S. Cl. CPC A61C 19/02; G06K 7/10386; G06K 19/0775; G06K 7/12; G06Q 10/087; A61C 8/00; A61B 2050/105

(57) ABSTRACT

A material management apparatus includes: an outer housing including a first left wall, a first right wall and a first rear wall; an inner housing including a second left wall, a second right wall, a front wall and a second rear wall; a top cover formed with an opening in which an antenna connector is disposed, wherein the antenna connector is connected to a RFID antenna which is connected to a RFID reader; and at least one case disposed in the inner housing, wherein each of the at least one case accommodates an object and the outer side thereof is equipped with a RFID tag; with the RFID tag, the RFID reader is able to count and manage the amount of objects being disposed in or taken out.

### BACKGROUND OF THE INVENTION

1. Field of the Invention

The present invention relates to material management apparatus, especially to a material management apparatus used for a dental material such as a dental implanting unit, in which an outer housing of the material management apparatus is installed with a RFID antenna and a case for storing the dental implanting unit is equipped with a RFID tag; with the RFID tag, a RFID reader at a remote location is able to count and manage the amount of objects being disposed in or taken out.

2. Description of Related Art

With the technology development, the technical level of dental implanting has been greatly improved, and the cost for dental implantation has been lowered to a level affordable to most of the consumers; as such, the dental implantation has become a second choice for a patient who needs denture.

However, the dental implanting unit is small in volume but high in price, a general dental implanting unit may cost hundreds of dollars, so the storage and management for dental implanting units are serious issues to be concerned.

Referring to FIG. 1, which is a schematic view illustrating a conventional storage case for dental implanting units. As shown in FIG. 1, the conventional storage case for dental implanting units includes an outer housing 100, an inner housing 110 and a top cover 120. The outer housing 100, the inner housing 110 and the top cover 120 are all made of stainless steel, and a dental implanting unit 150 is usually disposed in a case 160 having a foaming member, then the case 160 is disposed in the inner housing 110. When a dental implanting operation is desired to be processed, a dentist would take several dental implanting units 150 to let the patient to choose, after the patient makes his/her decision, the rest of the dental implanting units 150 are returned into the case 160 then the case 160 is disposed in the inner housing 110.

In the above-mentioned storage case for dental implanting units, functions such as checking whether the dentist returning the dental implanting units 150 after taken out and counting the quantity of the dental implanting units 150 disposed in the inner housing 110 are not provided; as such, a manual force is required for counting and checking the quantity of the dental implanting units, so a shortage of not being efficiency is caused.

### SUMMARY OF THE INVENTION

One primary objective of the present invention is to provide a material management apparatus, in which an outer housing thereof is installed with a RFID antenna and a case for storing a dental implanting unit is equipped with a RFID tag; with the RFID tag, a RFID reader at a remote location is able to count and manage the amount of objects being disposed in or taken out.

Another objective of the present invention is to provide a material management apparatus, in which an outer housing thereof includes a first left wall, a first right wall and a first rear wall, a first partition is formed between the first left wall, the first right wall and the first rear wall, and the width of the first partition is defined as one fourth to one sixth of the width of the first left wall and the width of the first right wall.

One another objective of the present invention is to provide a material management apparatus, in which an outer housing thereof includes a first left wall, a first right wall and a first rear wall, and the height of the first left wall and the height of the first right wall is defined as the half wavelength of a RFID wireless signal being added or subtracted by 2 cm.

Still one another objective of the present invention is to provide a material management apparatus, in which an inner housing thereof includes a second left wall, a second right wall, a front wall and a second rear wall, a second partition is formed between the second left wall, the second right wall and the second rear wall, and the width of the second partition is defined as one fourth to one sixth of the width of the second left wall and the width of the second right wall.

Still one another objective of the present invention is to provide a material management apparatus, in which an inner housing thereof includes a second left wall, a second right wall, a front wall and a second rear wall, and the height of the second left wall and the height of the second right wall is defined as the half wavelength of a RFID wireless signal being added or subtracted by 2 cm.

Accordingly, the present invention provides a material management apparatus, which includes: an outer housing including a first left wall, a first right wall and a first rear wall, wherein a first partition is formed between the first left wall, the first right wall and the first rear wall; an inner housing disposed in the outer housing and including a second left wall, a second right wall, a front wall and a second rear wall, wherein a second partition is formed between the second left wall, the second right wall and the second rear wall; a top cover capable of being covered on top of the outer housing and formed with an opening; an antenna connector disposed outside the opening, wherein the antenna connector is connected to a RFID antenna, and the RFID antenna is connected to a RFID reader; and at least one case disposed in the inner housing, wherein each of the at least one case accommodates an object and the outer side thereof is equipped with a RFID tag; with the RFID tag, the RFID reader is able to count and manage the amount of objects being disposed in or taken out.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic view illustrating a conventional storage case for dental implanting units;

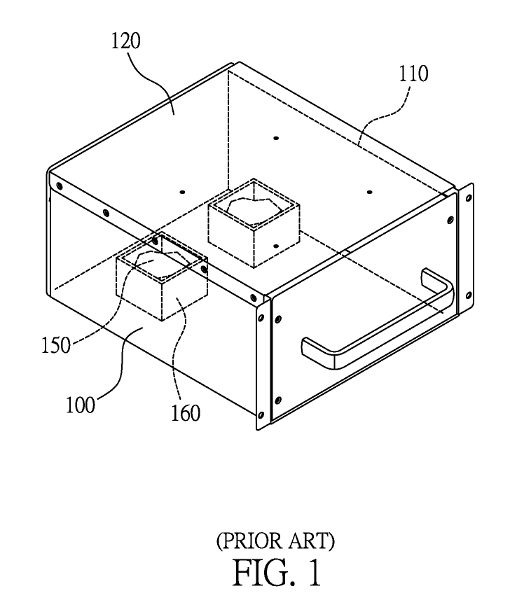

FIG. 2 is an exploded view illustrating the material management apparatus according to one preferred embodiment of the present invention;

FIG. 3 is a schematic view illustrating the assembly of the material management apparatus according to one preferred embodiment of the present invention; and

FIG. 4 is a schematic view illustrating the material management apparatus being connected to a RFID reader according to one preferred embodiment of the present invention.

### DETAILED DESCRIPTION OF THE PREFERRED EMBODIMENT

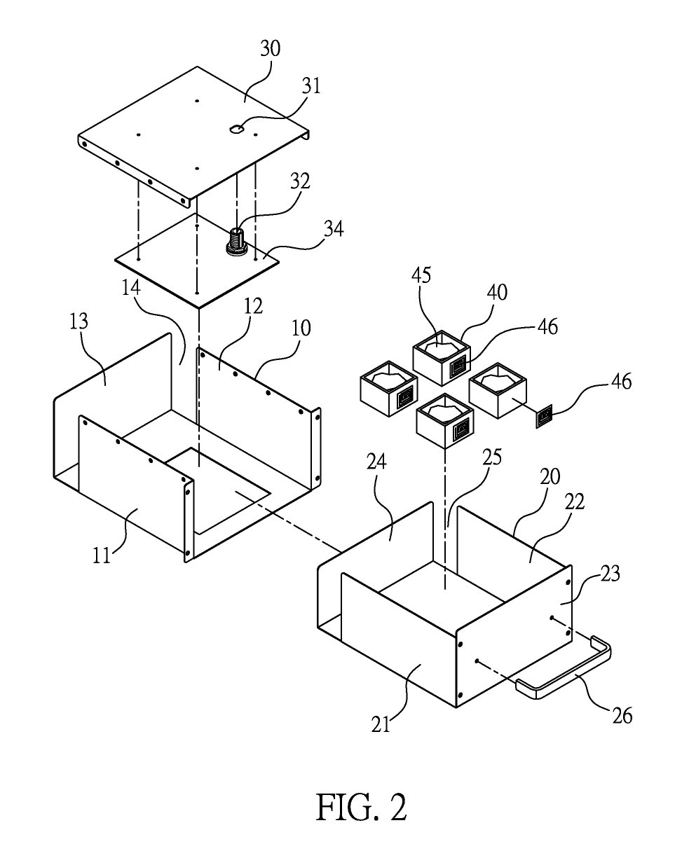

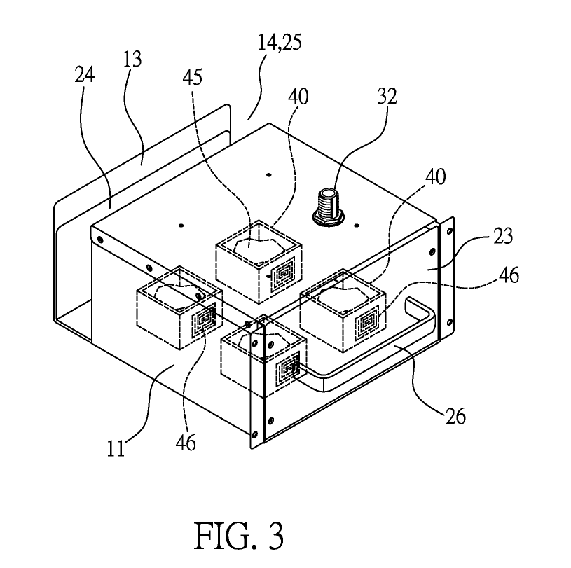

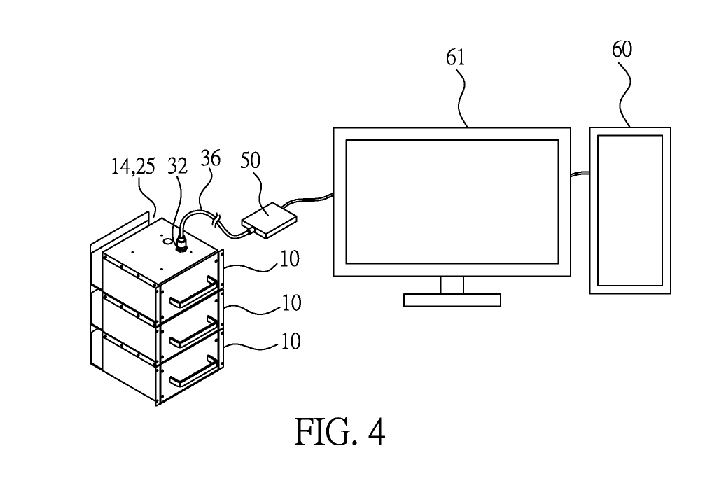

Referring from FIG. 2 to FIG. 4, wherein FIG. 2 is an exploded view illustrating the material management apparatus according to one preferred embodiment of the present invention; FIG. 3 is a schematic view illustrating the assembly of the material management apparatus according to one preferred embodiment of the present invention; and FIG. 4 is a schematic view illustrating the material management apparatus being connected to a RFID reader according to one preferred embodiment of the present invention.

As show in figures, the present invention provides a material management apparatus, which includes an outer housing 10, an inner housing 20, a top cover 30, an antenna connector 32 and at least one case 40.

The outer housing 10 is made of an electrical conductive material, e.g. but not limited to stainless steel, iron, copper and aluminum, and includes a first left wall 11, a first right wall 12 and a first rear wall 13, and a first partition 14 is formed between the first left wall 11, the first right wall 12 and the first rear wall 13 thereby allowing a RFID wireless signal to be emitted to the exterior, and the frequency of the RFID wireless signal is e.g. but not limited to 902˜928 MHz. Wherein, the width of the first partition 14 is e.g. but not limited to be defined as one fourth to one sixth of the width of the first left wall 11 and the width of the first right wall 12, and the height of the first left wall 11 and the height of the first right wall 12 is e.g. but not limited to be defined as the half wavelength of a RFID wireless signal being added or subtracted by 2 cm, for example when the frequency of the RFID wireless signal is 902˜928 MHz, the height of the first left wall 11 and the height of the first right wall 12 is 16 cm being added or subtracted by 2 cm.

The inner housing 20 is made of an electrical conductive material, e.g. but not limited to stainless steel, iron, copper and aluminum, deposed in the outer housing 10, and includes a second left wall 21, a second right wall 22, a front wall 23 and a second rear wall 24, and a second partition 25 is formed between the second left wall 21, the second right wall 22 and the second rear wall 24, thereby allowing the RFID wireless signal to be emitted to the exterior. Wherein, the width of the second partition 25 is e.g. but not limited to be defined as one fourth to one sixth of the width of the second left wall 21 and the width of the second right wall 22, and the height of the second left wall 21 and the height of the second right wall 22 is e.g. but not limited to be defined as the half wavelength of the RFID wireless signal being added or subtracted by 2 cm, for example when the frequency of the RFID wireless signal is 902˜928 MHz, the height of the second left wall 21 and the height of the second right wall 22 is 16 cm being added or subtracted by 2 cm.

The top cover 30 is made of an electrical conductive material, e.g. but not limited to stainless steel, iron, copper and aluminum, capable of being covered or fitted by screws for being fastened (not shown in figures) on top of the outer housing 10, and provided with a grounding function of conducting the outer housing 10 and the case 40, and the top cover 30 is formed with an opening 31.

The antenna connector 32 is exposed outside the opening 31, and the antenna connector 32 is connected to a RFID antenna 34, the RFID antenna 34 is able to emit or receive a RFID wireless signal, the frequency of the RFID wireless signal is 902˜928 MHz, and the antenna connector 32 is connected to a RFID reader 50 through a wire 36. The RFID reader 50 is able to be connected to one to eight RFID antennas 34 respectively through one to eight wires 36. Wherein, the RFID antenna 34 is formed on a printed circuit board (not shown in figures) through e.g. but not limited to a printing means, and the printed circuit board allows the antenna connector 32 to be fastened thereon, wherein the printed circuit board is formed with an antenna or the RFID antenna 34 through a printing means is a well-known prior art, therefore no further illustration is provided.

The at least one case 40 is disposed in the inner housing 20, each of the at least one case 40 accommodates an object 45, e.g. but not limited to a dental implanting unit, and the outer side thereof is equipped with a RFID tag 46. In addition, the interior of the case 40 is disposed with a foaming member (not shown in figures) for securing the object 45.

Moreover, according to the present invention, the front lateral wall of the inner housing 20 further comprises a handle 26, thereby enabling the inner housing 20 to be easily opened.

As shown in FIG. 2, when the material management apparatus of the present invention is desired to be assembled, the RFID antenna 34 is assembled with the antenna connector 32, then the top cover 30 is fitted by screws for being fastened on top of the outer housing 10; the RFID tag 46 is adhered at the outer side of the case 40, then the case 40 is disposed in the inner housing 20; lastly, the inner housing 20 is disposed in the outer housing 10; accordingly, the assembly of the material management apparatus of the present invention is finished. Wherein, the RFID reader 50 is able to provide the best sensing effect to the RFID tag 46 when the inner housing 20 is pushed into and fully enclosed by the outer housing 10.

As shown in FIG. 3, when the material management apparatus of the present invention is desired to be operated, the RFID antenna 34 is connected to the RFID reader 50 through the wire 36 and the connectable distance can be several meters or even longer, the RFID reader 50 is connected to a computer 60, the computer 60 is installed with an application program (APP, not shown in figures), the application program is able to count the quantity of the cases 40 disposed in the inner housing 20 and display the counting result on a display 61 of the computer 60. When a dental clinic personnel opens the inner housing 20 and takes out one case 40, the RFID reader 50 is able to timely sense the RFID tag 46 at the outer side of the case 40, and a signal is sent to the application program, so the application program is enabled to subtract one unit from the quantity of the objects 45, meanwhile the new result is shown on the display 61; or, when the dental clinic personnel opens the inner housing 20 and takes out four cases 40, the RFID reader 50 is able to timely sense the RFID tags 46 at the outer sides of the four cases 40, and a signal is sent to the application program, so the application program is enabled to subtract four units from the quantity of the objects 45 and the new result is shown on the display 61, after the patient makes his/her decision, the other three cases 40 is returned to the inner housing 20 by the dental clinic personnel and the inner housing 20 is pushed into and fully enclosed by the outer housing 10, the RFID reader 50 is able to timely sense the RFID tags 46 at the outer side of the three cases 40, and a signal is sent to the application program, so the application program is enabled to add three units to the quantity of the objects 45, meanwhile the new result is shown on the display 61; accordingly, the dental clinic personnel can use the application program to know the quantity of the objects 45 in the material management apparatus, so a daily inventory checking operation is not required.

In addition, because the outer housing 10 is formed with the first partition 14 and the inner housing 20 is formed with the second partition 25, the wireless signal having the frequency the 902˜928 MHz and emitted by the RFID antenna 34 is able to enter the inner housing 20 through the first partition 14 and the second partition 25 for sensing the RFID tag 46 then be sent back to the RFID reader 50, thereby enabling the application program to timely count the quantity of the objects 45 with respect to the numbers of RFID tags 46 sensed by the RFID reader 50. Accordingly, the material management apparatus provided by the present invention is novel and more practical in use comparing to prior art.

Based on what has been mentioned above for disclosing the material management apparatus provided by the present invention, the outer housing is installed with a RFID antenna, the case of the dental implanting unit is equipped with a RFID tag; with the RFID tag, the RFID reader at a remote location is able to count and manage the amount of objects being disposed in or taken out; the outer housing is formed with a first partition, the width of the first partition is defined as one fourth to one sixth of the width of the first left wall and the width of the first right wall, and the height of the first left wall and the height of the first right wall is defined as the half wavelength of the RFID wireless signal being added or subtracted by 2 cm; and the inner housing is formed with a second partition, the width of the second partition is defined as one fourth to one sixth of the width of the second left wall and the width of the second right wall, and the height of the second left wall and the height of the second right wall is defined as the half wavelength of the RFID wireless signal being added or subtracted by 2 cm. Accordingly, the present invention is novel and more practical in use comparing to prior art.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific examples of the embodiments disclosed and that modifications and other embodiments are intended to be included within the scope of the appended claims. Although specific terms are employed herein, they are used in a generic and descriptive sense only and not for purposes of limitation.

1. A material management apparatus, including:
an outer housing, including a first left wall, a first right wall and a first rear wall, wherein a first partition is formed between said first left wall, said first right wall and said first rear wall;
an inner housing, disposed in said outer housing and including a second left wall, a second right wall, a front wall and a second rear wall, wherein a second partition is formed between said second left wall, said second right wall and said second rear wall;
a top cover, capable of being covered on top of said outer housing and formed with an opening;
an antenna connector, exposed outside said opening, wherein said antenna connector is connected to a RFID antenna, and said RFID antenna is connected to a RFID reader; and
at least one case, disposed in said inner housing, wherein each of said at least one case accommodates an object and the outer side thereof is equipped with a RFID tag; with said RFID tag, said RFID reader is able to count and manage the amount of objects being disposed in or taken out.

2. The material management apparatus as claimed in claim 1, wherein said outer housing is an electrical conductive member, said inner housing is an electrical conductive member, and said top cover is an electrical conductive member.

3. The material management apparatus as claimed in claim 1, wherein the width of said first partition is defined as one fourth to one sixth of the width of said first left wall and the width of said first right wall.

4. The material management apparatus as claimed in claim 1, wherein said RFID antenna is able to emit or receive a RFID wireless signal, a frequency of said RFID wireless signal is 902˜928 MHz, and the height of said first left wall and the height of said first right wall is defined as a half wavelength of said RFID wireless signal being added or subtracted by 2 cm.

5. The material management apparatus as claimed in claim 1, wherein the width of said second partition is defined as one fourth to one sixth of the width of said second left wall and the width of said second right wall.

6. The material management apparatus as claimed in claim 1, wherein said RFID antenna is able to emit or receive a RFID wireless signal, a frequency of said RFID wireless signal is 902˜928 MHz, and the height of said second left wall and the height of said second right wall is defined as a half wavelength of said RFID wireless signal being added or subtracted by 2 cm.

7. The material management apparatus as claimed in claim 1, wherein the front wall of said inner housing further comprises a handle.

8. The material management apparatus as claimed in claim 1, wherein said object is a dental implanting unit.

9. The material management apparatus as claimed in claim 1, wherein said RFID antenna is formed on a printed circuit board through a printing means, and said printed circuit board allows said antenna connector to be fastened thereon.
